(19) United States
(12) Patent Application Publication
(10) Pub. No.: US 2024/0423520 A1
(43) Pub. Date: Dec. 26, 2024

(54) Funnel for Blood Collection into Small Format Container for Diagnostic Medical Testing

(71) Applicant: Blackfly Investments, LLC, DBA Molecular Testing Labs, Vancouver, WA (US)

(72) Inventors: Andrew Chastain, Vancouver, WA (US); Mariko Nakano, Vancouver, WA (US); Charles Sailey, Vancouver, WA (US); Adam Blackwell, Vancouver, WA (US); Peter Flynn, Vancouver, WA (US)

(21) Appl. No.: 18/214,334

(22) Filed: Jun. 26, 2023

(51) Int. Cl. A61B 5/15

(52) U.S. Cl. CPC A61B 5/150343

(57) ABSTRACT

A funnel for a small-format blood collection device and system according to embodiments described herein. The system may include a funnel having a first conical portion attached to a second cylindrical portion. At the confluence of these two portions is a shoulder having a raised exterior contour to match a contour of a mating small-format container. The mating of the contours of the shoulder and a matching topside of a small-format container provides for greater stability when utilizing the funnel during a blood collection procedure by way of capillary bleeding.

### BACKGROUND

Small format blood containers (sometimes called Microtainers®) are useful for collecting blood into a container to be used in diagnostic medical testing at some later time after collection. These small format containers typically have a smaller overall size than normal-sized blood collection containers to assist in facilitating shipping and handling when blood samples are required for remote analysis and diagnostics. That is, a small format container is easier to send through the mail or shipping companies due to the smaller format. As such, remote testing and diagnostics are more readily accessible when using small format containers for blood collection.

However, capturing blood with accuracy and without contamination or mess is difficult due to the small format nature of these collection containers. That is, these small format containers have a relatively small opening at the top of the collection tube into which a user must accurately drip a blood sample. Further, balancing a narrow small-format collection tube may be precarious as any slight touch may unbalance the collection tube further exacerbating the collection process and any spillage that may result. Thus, a small-format blood collection tube can be fraught with difficulties in aiming a blood drip and taking care to maintain the collection without tipping, spilling and/or contamination.

### BRIEF DESCRIPTION OF THE DRAWINGS

Embodiments of the subject matter disclosed herein in accordance with the present disclosure will be described with reference to the drawings, in which:

FIG. 1 is an isometric diagram of a funnel suited to interface with a small-format blood collection container according to an embodiment of the subject matter disclosed herein;

FIG. 2 is another view of the funnel 100 from FIG. 1 shown near with a small-format container 200 according to an embodiment of the subject matter disclosed herein;

FIG. 3 is a diagram of the funnel of FIGS. 1 engaged with a small-format blood collection container of FIG. 2 according to an embodiment of the subject matter disclosed herein; and

FIG. 4 is a diagram illustrating the use of the funnel of FIG. 1 according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

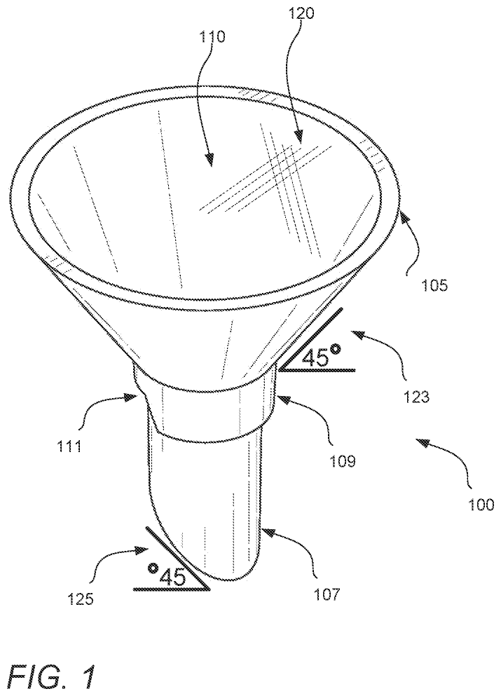

By way of an overview, the systems and devices discussed herein may be directed to a funnel for a small-format blood collection device and system according to embodiments described herein. The system may include a funnel having a first conical portion attached to a second cylindrical portion. At the confluence of these two portions is a shoulder having a raised exterior contour to match a contour of a mating small-format container. The mating of the contours of the shoulder and a matching topside of a small-format container provides for greater stability when utilizing the funnel during a blood collection procedure by way of capillary bleeding. This is particularly useful for “at-home” blood collections to be used in remote diagnostics and testing. These and other aspects of the subject matter disclosed herein are described below with respect to FIGS. 1-4.

FIG. 1 is an isometric diagram of a funnel 100 suited to interface with a small-format blood collection container (not shown in FIG. 1) according to an embodiment of the subject matter disclosed herein. As is known, a funnel is typically a tube or pipe that is wide at the top and narrow at the bottom and used for guiding liquid or powder into a small opening, such as a tank or container. In this sense, the funnel 100 of FIG. 1 accomplished this task with wide top portion 105 that is conical with a larger opening aperture 110 intended to collect blood for a blood collection procedure. Additionally, the aperture leads to a narrowing of the conical portion 105 that guides liquid or powder to an opening at the base of the conical section 105 that is attached to a bottom portion 107 that is cylindrical with its own aperture (unseen at the interior bottom of the top portion 105) that remains a substantially consistent diameter throughout cylindrical section 107. In the funnel of FIG. 1, this embodiment also includes a shoulder portion 109 attached surrounding the cylindrical portion 107 at a confluence of the conical portion 105 and the cylindrical portion 107. Further, this shoulder 109 (sometimes called a collar) includes an exterior contour 111 to match a contour of a mating small-format container (not shown here).

The funnel 100 of FIG. 1 may be made of stainless steel, aluminum, glass, or plastic. The material used in its construction should be sturdy enough to withstand the weight of the substance being transferred, and it should not react with the substance. In one embodiment, the funnel 100 is made from a medical grade random copolymer polypropylene material (P5M6K-048) and exhibits an SPI-A2 (Society of the Plastic Industry) finish 120 that is polished on the inside face of the top portion 105 to assist with moving blood down the funnel 100 into container (not shown). SPI gloss grades are suitable for products with a smooth surface finish for cosmetic and functional reasons. For example, A2 is the most common diamond finish used in industry, resulting in visually pleasing surfaces with a suitable viscosity and release. In addition, grade “A” surface finishes are used on optical parts such as lenses, mirrors, and medical equipment. The combination of a low surface roughness (SPI-A2 finish) with a low surface energy plastic (like polypropylene or polyethylene) reduces the ability of blood to wet (e.g., stick to) the surface of the funnel, thus reducing the blood retained by the funnel. In FIG. 1, this A2 finish is shown only as portion of the interior surface and in dotted line format for the ease of illustration. In other embodiments, the plastic funnel 100 is made from a non-reactive polyethylene material or other low-surface energy plastic.

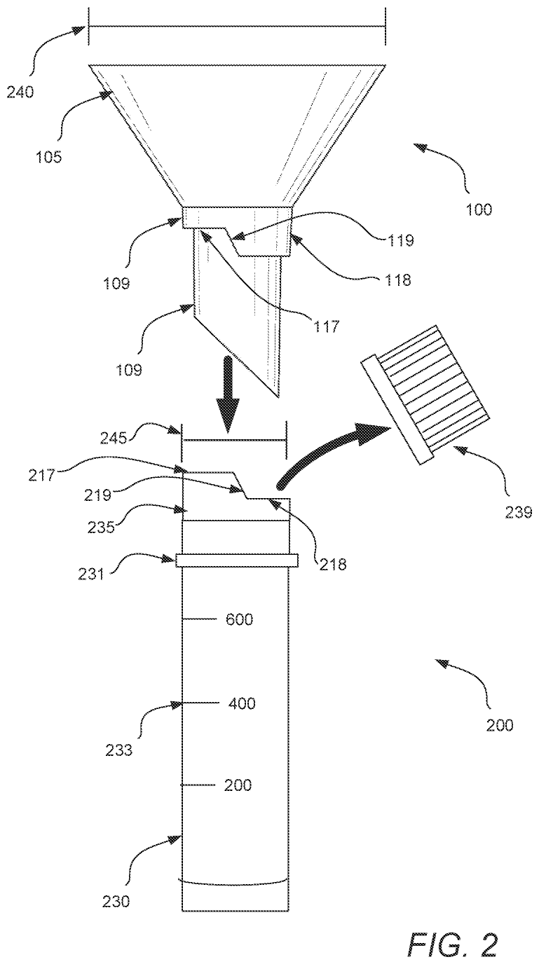

In still further embodiments, the funnel 100 of FIG. 1 includes a 45-degree angle 123 for the conical section 105. In other embodiments, this angle may vary between 30 and 60 degrees and assists with weight balance while yielding optimal collection orifice. As this conical angle 123 is reduced, any risk from tipping (when mated with a small-format container) may also be reduced. A 45-degree angle is one way to achieve a suitable size collection aperture while reducing the risk of tipping from any overly wide funnel. Further yet, the cylindrical portion may also have a bottom side aperture that exhibits a 45-degree chamfer 125 from the major axis of the cylindrical section. This chamfer angle 125 assist with creating a point-like tip that makes the funnel 100 easier to mate with an aperture of a small-format container (not shown). Additionally, the 45-degree chamfer angle 125 on bottom of spout assists in directing blood to a single point on the spout which both facilitates transfer into the collection device and minimizes the volume of blood retained by the spout. In one embodiment, the conical shaped portion has a top end aperture having an opening at least three times as large in diameter as an aperture opening of the conical shaped portion. The system that includes a small-format container is discussed next with respect to FIG. 2.

FIG. 2 is another view of the funnel 100 from FIG. 1 shown near with a small-format container 200 according to an embodiment of the subject matter disclosed herein. As discussed above, the funnel 100 includes a top conical portion 105 attached to a bottom cylindrical portion 107 with a contoured shoulder portion 109 attached at the confluence of the top conical portion 105 and the bottom cylindrical portion 107. The funnel 100 is designed to interface with a small-format container 200 having a contour that is a mirror image of the contour of the shoulder portion 109. In this sense, the funnel 100 may “lock-into” place with the small-format container 200 thereby exhibiting a seemingly integrated blood collection device to used primarily with a finger-prick blood collection procedure. The small-format container 200 may have graduated markings 233 on a collection tube 230 that may have a top-side rim 231 suited to securely interface with a removable cap 239. When the funnel 100 is mated with the small-format container 200, the funnel 100 may be used to assist with a blood collection procedure in that the conical portion 105 exhibits a larger diameter aperture 240 than the diameter of the top aperture 245 of the small-format container 200. In one embodiment the aperture 240 is three time larger in diameter than the aperture 245.

In systems of the past, sometimes a person collecting a blood sample (without benefit of a securely-mating funnel 100) would choose to use only the small-format container 200 to “catch” capillary bleeding form a finger prick. This often led to spillage and loss of sample. Furthermore, a user may be tempted to use the small-format container to scoop spilled blood sample that may lead to loss of sample and contamination of the sample. The securely mated funnel 100 assists with whole sample collection and reduction of contamination possibilities. Further yet, utilizing the securely mated funnel 100 allows for faster and cleaner blood collection that mitigates problems from faster clotting time from finger pricks.

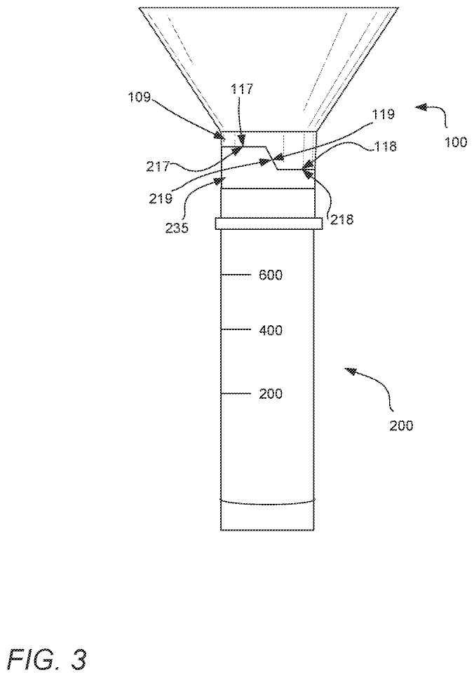

The matching contours of the shoulder portion 109 of the funnel 100 and the top contour 235 of the small-format container 200 are designed in a manner to both assist with maintaining a mated position between the funnel 100 and the small-format container 200 as well as assisting a process for releasing the funnel 100 from the small-format container 200 after blood collection. As can be seen, the contour of the shoulder portion 109 of the funnel has a first flat portion 117 disposed at a first level and second flat portion 118 disposed at a second level with an angled portion 119 between these two flat portions. Similarly, but in a mirror-image manner, the top contour portion 235 of the small-format container 200 also includes a first flat portion 217 disposed at a first level and second flat portion 218 disposed at a second level with an angled portion 219 between these two flat portions. In this manner, when fully secured and mated, the first flat portion 117 of the shoulder portion 109 is contiguous with the first flat portion 217 of the top-portion contour 235 of the small-format container 200. Similarly, the second flat portion 118 of the shoulder portion 109 is contiguous with the second flat portion 218 of the top-portion contour 235 of the small-format container 200. Additionally, the angled portion 119 of the shoulder portion 109 is contiguous with the angled portion 219 of the top-portion contour 235 of the small-format container 200. The mating of these two portions is illustrated in FIG. 3 and discussed below.

FIG. 3 is a diagram of the funnel 100 of FIG. 1 engaged with a small-format blood collection container 200 of FIG. 2 according to an embodiment of the subject matter disclosed herein. When mated, one can see that flat portions 117 and 217 are mated contiguous with each, flat portions 118 and 218 are mated contiguous with each other, and angled portions 119 and 219 are mated contiguous with each other. This mating helps provide stability when the funnel is mounted to and engaged with the small-format container 200. As alluded to above, if one were to twist the funnel portion 100 or the small-format container 200 either clockwise or counter-clockwise with respect to a major axis of the small-format container 200, the angled portions 119 and 219 would push against each other to force the funnel 100 and the small-format container 200 away from each other. That is, eventually the lower flat portion 118 of the shoulder 109 of the funnel would be forced to the level of the top portion 217 of the small-format container 200. This assists with breaking any static friction force that may be biasing the funnel 100 and the small-format container 200 together. It does so in a controlled manner mitigating opportunities for spillage or dropping any collected blood in the small-format container 200.

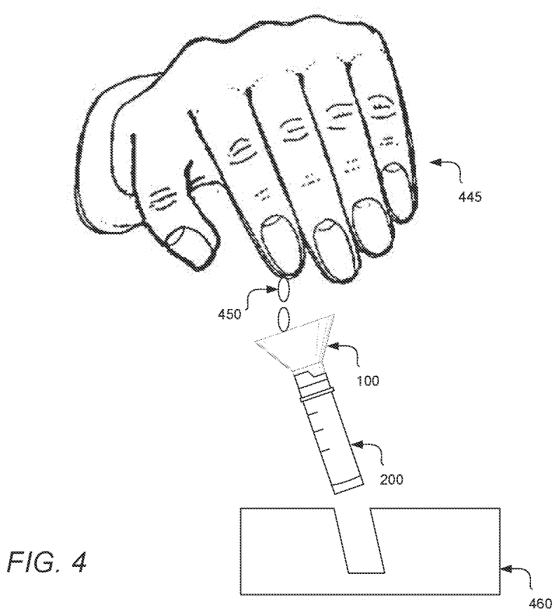

FIG. 4 is a diagram illustrating the use of the funnel 100 of FIG. 1 according to an embodiment of the subject matter disclosed herein. In this illustration, one can see that a human hand 445 has been pricked (at the index finger pad) to produce capillary bleeding 450 that may be collected by the funnel 100 that is attached to a small-format container 200 in a manner discussed above with respect to FIG. 3. As the funnel 100 has a suitable top-end aperture, it becomes easier for the capillary bleeding 450 to be collected into the small-format container 200. This helps mitigate any need to scoop blood at the finger prick or problems associated with contamination if small portions of the sample come into contact with the outside of the small-format container 200 and subsequently “pushed” back into the small-format container 200 by foreign contact (e.g., a human hand, the cap of the small-format container, or any other object).

In an effort to assist with holding the small-format container in a vertical position while one is pricking one's finger (presumably with one's other hand), the system may also include a small holding device 460 that is suited to assist with maintaining a vertical or close-to-vertical position of the small format container while engaged with the funnel 100. In one embodiment, the holding device 460 is a foldable triangular shape having an orifice 465 at or near a top portion just large enough to insert a small-format container to about a halfway point in the height of the small format container. In other embodiment, the space of the holder may be different with differing depths of the orifice 465.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

That which is claimed is:

1. A funnel for interfacing with a small-format blood collection container, the funnel comprising:
a first portion having a conical shape;
a second portion having a cylindrical shape with a major axis; and
a shoulder attached surrounding the cylindrical-shaped portion at a confluence of the conical-shaped portion and the cylindrical portion, the shoulder shaped to match a contour of a mating small-format container.

2. The funnel of claim 1, wherein conical-shaped portion comprises side walls disposed at 45 degrees with respect to the major axis.

3. The funnel of claim 1, wherein a bottom end of the cylinder-shaped portion comprises a 45-degree chamfer

4. The funnel of claim 1, wherein conical shaped portion comprises a top end aperture having an opening at least three times as large in diameter as an aperture opening of the conical-shaped portion.

5. The funnel of claim 1, further comprising a polypropylene material.

6. The funnel of claim 1, wherein the conical-shaped portion comprises a height dimension that is approximately equal to a height dimension of the cylinder-shaped portion.

7. The funnel of claim 1, further comprising a conical-shaped portion surface with an SPI-A2 rated surface.

8. The funnel of claim 1, wherein the shoulder contour shape comprises:
a first shoulder portion having a first flat edge disposed at a first horizontal level;
a second shoulder portion having a second flat edge disposed at a second horizontal level that is different than the first horizontal level; and
a third shoulder portion that is angled between the first shoulder portion and the second shoulder portion.

9. The funnel of claim 1, wherein the cylinder-shaped portion of the funnel comprises an outer diameter suited to be inserted into a small-format container.

10. The funnel of claim 1, further comprising wherein the cylinder-shaped portion of the funnel comprises an outer diameter suited to be inserted into an industry-standard microtainer.

11. A system for facilitating capillary blood collection, the system comprising:
a small-format blood collection container having a cylindrical top-end aperture with a contoured edge; and
a funnel for interfacing with the small-format blood collection container, the funnel including:
a first portion shaped conically;
a second portion shaped cylindrically having a major axis; and
a shoulder attached surrounding the cylindrical portion at a confluence of the conical portion and the cylindrical portion, the shoulder shaped to match the contoured edge of the top-end aperture of the small-format blood collection container.

12. The system of claim 11, further comprising a holding device suited to hold the small-format blood collection container upright during a capillary blood collection procedure.

13. The system of claim 11, further comprising a cap suited to interface with the top-end aperture of the small-format blood collection container

14. The system of claim 11, wherein the small format container comprises an industry-standard microtainer.

15. The system of claim 11, wherein conically shaped portion comprises side walls disposed at an angle between 30 degrees and 60 degrees with respect to the major axis.

16. The system of claim 11, wherein a bottom end of the cylindrical shaped portion comprises a chamfer having and angle between 30 degrees and 60 degrees

17. The system of claim 11, wherein conically shaped portion comprises a top end aperture having an opening at least three times as large in diameter as an aperture opening of the conically shaped portion.

18. The system of claim 11, wherein the funnel and the small-format container comprise a polypropylene material.

19. The system of claim 11, wherein:
the shoulder contour shape comprises:
a first shoulder portion having a first flat edge disposed at a first horizontal level suited to interface contiguously with a first flat edge of the contour of the top-end of the small-format container at the first horizontal level;
a second shoulder portion having a second flat edge disposed at a second horizontal level suited to interface contiguously with a second flat edge of the contour of the top-end of the small-format container at the second horizontal level; and
a third shoulder portion that is angled between the first shoulder portion and the second shoulder portion and suited to interface contiguously with third portion of the contour of the top-end of the small-format container between the first horizontal level and second horizontal level.

20. The system of claim 11, wherein the funnel comprises an SPI-A2 rating.
